(19) United States
(12) Patent Application Publication
(10) Pub. No.: US 2023/0091118 A1
(43) Pub. Date: Mar. 23, 2023

(54) FLEXIBLE URINE COLLECTION CONTAINER AND RELATED SYSTEMS AND METHODS

(71) Applicant: PureWick Corporation, El Cajon, CA (US)

(72) Inventor: Kristin Louise Watson, Social Circle, GA (US)

(21) Appl. No.: 17/933,590

(22) Filed: Sep. 20, 2022

Related U.S. Application Data
(60) Provisional application No. 63/247,375, filed on Sep. 23, 2021.

(51) Int. Cl. A61B 10/00; A61F 5/44

(52) U.S. Cl. CPC A61B 10/007; A61F 5/4404

(57) ABSTRACT

Examples relate to urine collection container as a flexible bladder and baffles, and related systems and methods. A urine collection system includes a urine collection device, a conduit, a flexible bladder, and a baffle structure. The urine collection device is configured to be positioned at least proximate to a urethra of a user. The conduit is in fluid communication with the urine collection device. The flexible bladder has an interior region, and the baffle structure is positioned or positionable in the interior region of the bladder.

### CROSS-REFERENCE TO RELATED APPLICATIONS

This application claims priority to U.S. Provisional Patent Application No. 63/247,375 filed on Sep. 23, 2021, the disclosure of which is incorporated herein, in its entirety, by this reference.

### BACKGROUND

An individual may have limited or impaired mobility such that typical urination processes are challenging or impossible. For example, the individual may have surgery or a disability that impairs mobility. In another example, the individual may have restricted travel conditions such as those experienced by pilots, drivers, and workers in hazardous areas. Additionally, fluid collection from the individual may be needed for monitoring purposes or clinical testing.

Bed pans and urinary catheters, such as a Foley catheter, may be used to address some of these circumstances. However, bed pans and urinary catheters have several problems associated therewith. For example, bed pans may be prone to discomfort, spills, and other hygiene issues. Urinary catheters be may be uncomfortable, painful, and may cause urinary tract infections. Conventional urine collection devices also may be limited to use when a patient is confined to a bed in a supine position.

Thus, users and manufacturers of fluid collection devices continue to seek new and improved devices, systems, and methods to collect urine.

### SUMMARY

Embodiments disclosed herein are related to a urine collection system with a flexible bladder collection container for noise mitigation, and related systems and methods. In an embodiment, a urine collection system includes a urine collection device, a conduit, a flexible bladder, and a baffle structure. The urine collection device is configured to be positioned at least proximate to a urethra of a user. The conduit is in fluid communication with the urine collection device. The flexible bladder includes an interior region in fluid communication with the conduit and the baffle structure is positioned or positionable in the interior region of the canister.

In an embodiment, a urine collection container includes a flexible bladder, a baffle structure, and a port. The flexible bladder has an interior region and the baffle structure includes one or more baffles disposed within the interior region of the canister. The port is secured or securable to the bladder and includes a collection connector and a vacuum connector.

In an embodiment, a method of collecting urine includes positioning a fluid collection device at least proximate to a urethra of a user, the urine collection device being in fluid communication with a flexible bladder through a conduit. The method also includes collecting urine discharged by the user in the urine collection device. The method also includes drawing urine from the urine collection device through the conduit into the interior region of the bladder, the urine contacting at least a portion of the baffle structure in the interior region of the bladder.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is a block diagram of a portable urine collection system, according to an embodiment.

FIG. 1B is an isometric front view of a urine collection system, according to an embodiment.

FIG. 2A is a top isometric view of a flexible bladder for a urine collection system, according to an embodiment.

FIG. 2B is a cross-sectional view of a vacuum connector, according to an embodiment.

FIG. 3A is a cross-sectional view of an interior of a flexible bladder showing a baffle structure for a urine collection system, according to an embodiment.

FIG. 3B is a cross-sectional view of an interior of a flexible bladder showing a baffle structure for a urine collection system, according to an embodiment.

FIG. 3C is a cross-sectional top view of an interior of a flexible bladder showing a baffle structure for a urine collection system, according to an embodiment.

FIG. 4 is a flow diagram of a method for collecting urine, according to an embodiment.

### DETAILED DESCRIPTION

Embodiments disclosed herein are related to urine collection containers and systems including baffle structures, and related methods. The devices and systems disclosed herein are configured to collect fluids from an individual. The fluids collected by the fluid collection devices may include at least one of urine, vaginal discharge, penile discharge, reproductive fluids, blood, sweat, or other bodily fluids.

In conventional urine collection systems, the sound of urine splashing during urine collection and/or canister movement can often embarrassing for users. For example, the urine may enter at the top of a canister and fall to the bottom of the canister or the urine level in the canister, increasing the splashing noise as the falling urine increases speed before contacting the bottom of the container or the urine level. The splashing noise may draw attention to the urine collection system during urine collection and/or movement by the user. Users of urine collection systems may have concerns about discretion when using urine collection systems, especially when the urine collection systems are used in ambulatory settings or other settings outside of a clinical setting. For example, some users may use a urine collection system while sitting in a wheelchair, with the urine collection canister positioned on a support on the wheelchair. As the wheelchair moves, the urine in the canister may create an embarrassing splashing noise, preventing the discretion desired by the user.

To improve conventional urine collection systems, the urine collection system can include a flexible bladder. The bladder can include baffle structures described herein positioned or positionable in the flexible bladder of urine collection systems. In some embodiments, the baffle structure may be a foam structure inserted into the bladder to act as a baffling system that prevents the urine (or other fluids) from sloshing and/or splashing within the bladder, thereby preventing or inhibiting the distinct splashing noise common to conventional urine collection systems. In some embodiments, the baffle structure can include a ridge and/or series of ridges disposed on an interior surface of the bladder. The baffle structure may be configured to create a tortuous path for the urine to flow through the baffle structure within the flexible bladder and mitigate the noise of the fluid.

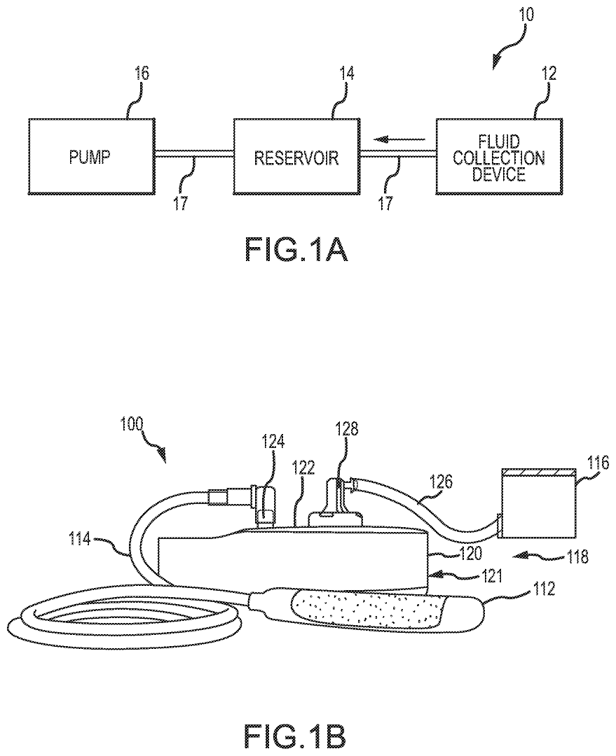

FIG. 1A is a block diagram of a fluid collection system 10, according to an embodiment. The fluid collection system 10 may be included in embodiments of fluid collection systems described herein. The system 10 includes a fluid collection device 12 (e.g., any of the fluid collection devices disclosed herein), a reservoir 14 (e.g. a flexible bladder), and a pump 16 (or vacuum device). The fluid collection device 12, the reservoir 14, and the pump 16 may be fluidly coupled to each other via one or more conduits 17. For example, fluid collection device 12 may be operably coupled to one or more of the reservoir 14 or the pump 16 via the conduit 17. In some embodiments, the pump 16 may be secured directly to the reservoir 14. Fluid (e.g., urine or other bodily fluids) collected in the fluid collection device 12 may be removed from the fluid collection device 12 via the conduit 17 secured to the fluid collection device 12. Suction force may be introduced into the chamber of the fluid collection device 12 via the inlet of the conduit 17 responsive to suction (e.g., vacuum) force applied at the outlet of the conduit 17.

The suction force may be applied to the outlet of the conduit 17 by the pump 16 either directly or indirectly. The suction force may be applied indirectly via the reservoir 14. For example, the outlet of the conduit 17 may be disposed within or fluidly coupled to an interior region of the reservoir 14 and an additional conduit 17 may extend from the reservoir 14 to the pump 16. Accordingly, the pump 16 may apply suction to the fluid collection device 12 via the reservoir 14. The suction force may be applied directly via the pump 16. For example, the outlet of the conduit 17 may be disposed within the pump 16. An additional conduit 17 may extend from the pump 16 to a point outside of the fluid collection device 12, such as to the reservoir 14. In such examples, the pump 16 may be disposed between the fluid collection device 12 and the reservoir 14.

The reservoir 14 is sized and shaped to retain a fluid therein. The reservoir 14 may include a flexible bladder (e.g., drainage bag), an expandable pouch, or any other enclosed flexible container for storing bodily fluid(s) such as urine. In some examples, the conduit 17 may extend from the fluid collection device 12 and attach to the reservoir 14 at a first point therein. An additional conduit 17 may attach to the reservoir 14 at a second point thereon and may extend and attach to the pump 16. Accordingly, a vacuum (e.g., suction) may be drawn through fluid collection device 12 via the reservoir 14. Fluid, such as urine, may be drained from the fluid collection device 12 using the pump 16.

The pump 16 may include one or more of a manual vacuum pump, and electric vacuum pump, a diaphragm pump, a centrifugal pump, a displacement pump, a magnetically driven pump, a peristaltic pump, or any pump configured to produce a vacuum. The pump 16 may provide a vacuum or suction to remove fluid from the fluid collection device 12. In some examples, the pump 16 may be powered by one or more of a power cord (e.g., connected to a power socket), one or more batteries, or even manual power (e.g., a hand operated vacuum pump). In some examples, the pump 16 may be sized and shaped to fit outside of, on, or within the fluid collection device 12. For example, the pump 16 may include one or more miniaturized pumps or one or more micro pumps. The vacuum sources disclosed herein may include one or more of a switch, a button, a plug, a remote, or any other device suitable to activate the pump 16.

FIG. 1B shows an example of a urine collection system 100, according to an embodiment. The urine collection system 100 may include a urine collection device 112, a first conduit 114 in fluid communication with an interior region of the urine collection device 112, a pump 116, and a urine collection container 118. The urine collection container 118 may include a flexible bladder 120 that includes an expandable pouch 121 and a port 122 secured or securable to the expandable pouch 121. The bladder 120 may include a top region and a bottom region, and may at least partially define an interior region in fluid communication with the internal region of the urine collection device 112 via the first conduit 114. For example, the port 122 may include a collection connector 124 to which the first conduit 114 may be detachably secured to provide fluid communication between urine collection device 112 and the interior region of the flexible bladder 118. A second conduit 126 may provide fluid communication between the interior region of the flexible bladder 120 and the pump 116. For example, the port 122 may include a vacuum connector 128 to which the second conduit 126 may be detachably secured to provide fluid communication between the pump 116 and the interior region of the flexible bladder. The vacuum connector 128 and/or the collection connector 124 may include an adapter detachably secured thereto to secure the respective conduit 114, 126 to the vacuum connector 128 and/or the collection connector 124. In some embodiments, at least one (e.g., both) of the first conduit 114 and the second conduit 126 may be detachably connected or secured to the port 122 effective to provide fluid communication with the interior region of the flexible bladder 120. For example, the port 122 may include at least one (e.g. both) of the collection connector 124 and the vacuum connector 128.

The urine collection device 112 may be configured to be positioned at least proximate to a urethra of a user. While the urine collection device 112 shown in FIG. 1B includes a female urine collection device, the urine collection device 112 may instead include a male urine collection device. PCT International Application No. PCT/US2019/029616, for example, describes various embodiments of both male and female fluid collection devices, the disclosure of which are incorporated in their entirety by this reference. Moreover, the urine collection device 112 may be interchangeable in the urine collection system 100 between different types, varieties, and sizes of male or female urine collection devices. Generally, the urine collection device 112 may include a surface sized to be positioned proximate or adjacent to the urethra and configured to wick urine or other fluids away from the user. Urine or other fluids may be wicked from the surface to a reservoir in the urine collection device 112.

The urine collection system 100 also includes the first conduit 114 in fluid communication with an interior region (e.g. reservoir) of the urine collection device 112 and the interior region of the urine collection container 118. The first conduit 114 may be positioned between the urine collection device 112 and the urine collection container 118. The urine collection system 100 also may include the second conduit 126 providing fluid communication between the pump 116 and the interior region of the flexible bladder 120. In some embodiments, the pump 116 may be secured directly to the urine collection container 118, and the second conduit 126 may be absent from the urine collection system 100. The conduits 114, 126 may include a flexible tube. In some embodiments, at least a portion of the first conduit 114 may be substantially opaque, thereby inhibiting viewing of the urine within the first conduit 114. In some embodiments, the first conduit 114 and/or the second conduit 126 can include a small interior diameter to further mitigate noise of the urine passing there through. In some embodiments, the first conduit 114 and/or the second conduit 126 may include an interior diameter between about 0.3 inches and about 0.2 inches. In some embodiments, the first conduit 114 and/or the second conduit 126 may include an interior diameter of about 0.25 inches.

The flexible bladder 120 may be disposable and may include a generally flexible material such as one or more of polyethylene, thermoplastic elastomers, or poly vinyl chloride (PVC). In some embodiments, the bladder 120 can include a silicone rubber. In some embodiments, the flexible bladder 120 may be drained and reused. The urine collection system 100 may be configured to accommodate different volumetric sizes of bladders 120. The flexible bladder 120 may be substantially flat and expandable as it fills with fluid. In some embodiments, the flexible bladder 120 may include other shapes and configurations, such as a rectangular side profile. In some embodiments, the port 122 may include multiple ports and/or adapters for attachment of the conduits 114, 126 to the flexible bladder 120. In some embodiments, the port 122 may include additional connectors or adapters than those shown in FIG. 1B. Except for the ports and/or adapters for attachment of the conduits 114, 128, the flexible bladder 120 may be sealed and airtight such that the urine collected in the canister 114 does not leak or spill.

The pump 116 may be in fluid communication with the interior region of the flexible bladder 120 and is configured to pull a vacuum on the interior region of the flexible bladder 120 effective to draw the urine from the urine collection device 112 through the first conduit 114 into the flexible bladder 120. The pump 116 may be secured directly to the urine collection container 118, or the conduit 126 may fluidly couple the pump 116 with the interior region of the flexible bladder 120. The pump 116 may include one or more of a manual vacuum pump, and electric vacuum pump, a diaphragm pump, a centrifugal pump, a displacement pump, a magnetically driven pump, a peristaltic pump, or any pump configured to produce a vacuum. The pump 116 may provide a vacuum or suction to remove fluid from the fluid collection device 112. In some examples, the pump 116 may be powered by one or more batteries or other power sources. The pump 116 may be included in a housing that also supports the urine collection container 118. In some examples, the pump 116 may be sized and shaped to fit within a container support on a wheelchair. In some embodiments, the pump 116 may include a wall-mounted pump. The pump 116 may include one or more of a switch, a button, a plug, a remote, or any other device suitable to activate the pump 116.

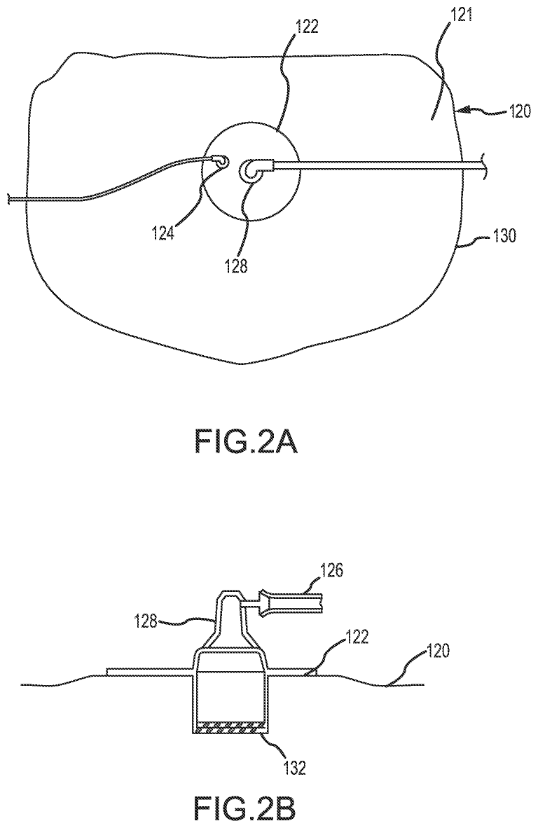

FIG. 2A is a top isometric view of a urine collection container 118 including the flexible bladder 120 and the port 122 coupled to the top of the flexible bladder 120. The flexible bladder 120 can include the expandable pouch 121 and a baffle structure positioned in the interior region of the flexible bladder 120, according to an embodiment. In some embodiments, the expandable pouch 121 can include a first and second sheet of material sealed together in selected areas to form one or more edge boundary 130 of the interior of the flexible bladder 120. The edge boundary 130 may be formed by heat sealing the first and second sheets together or by applying an adhesive to hold the sheets together in the desired regions. One edge of the flexible bladder 120 may be formed by folding a sheet over upon itself (e.g., center fold) such that a seal is not required to form the corresponding edge that in essence forms a demarcation between the first and second sheets.

In some embodiments, the expandable pouch 121 may include polymeric films or fibrous sheets. Useful polymeric films may comprise one or more thermoplastic materials selected from polyolefins, for example, polyethylene homopolymers, such as low density polyethylene (LDPE) and high density polyethylene (HDPE), or polyethylene copolymers. The polymeric film may be monolayer or multilayer, for example, a three-layer film having a sealant layer, a core layer, and an outer durable layer.

In some embodiments, the port 122 may be positioned generally on an upper exterior surface of the bladder 102. The port 122 may be positioned on the upper surface of the bladder to prevent fluid from entering the vacuum connector 128. The positioning of the port 122 can ensure that the urine collection system 100 maintains vacuum to draw urine from the fluid collection device 112. In some embodiments, the port 122 can include both the collection connector 124 and the vacuum connector 128. In some embodiments, the collection connector 124 and the vacuum connector 128 may be separate and attach to the flexible bladder 120 at other suitable locations. In some embodiments, the port 122 can include a drainage connector, a vent, a spout, or other suitable connector or device that allows access to the interior of the flexible bladder 120.

FIG. 2B is a cross-sectional view of a vacuum connector 128, according to an embodiment. In some embodiments, the vacuum connector 128 may include a membrane 132 configured to prevent fluid from traversing the vacuum connector 128. The membrane 132 can include a material such as a Polytetrafluoroethylene fibers mesh that allows air to pass through but not fluids. The membrane 132 can include any suitable hydrophobic material and/or a hydrophobic coating. The membrane 132 can include a thickness of about 40 μm to about 200 μm. In some embodiments, the membrane 132 can include a pore size between about 5 μm to about 50 μm. In some embodiments, the membrane 132 may include silicone.

In some embodiments, the urine collection system 100 may include a baffle structure positioned or positionable in the interior region of the flexible bladder 120. The baffle structure may include one or more baffles configured to improve discretion during urine collection and/or filling of the flexible bladder 120. For example, in contrast to conventional urine collection system, the baffle structure may prevent urine or other fluid from splashing into the flexible bladder 120 like would occur with a rigid canister as the urine is drawn from the user and dispensed into the flexible bladder 120. In conventional systems, urine splashing into a canister draws attention to the urine collection system due to the noise generated by the urine splashing in the canister during and after filling of the canister. In urine collection systems designed for ambulatory patients, noise from urine splashing during collection and movement is increased and/or causes additional embarrassment for users.

To improve the urine collection system 100, a baffle structure may be positioned or positionable in the interior region of the flexible bladder 120. The baffle structure may reduce the distance urine can move within the flexible bladder 120, thereby inhibiting amplification of the splashing noise. To reduce the distance the urine is able to move and/or slosh within the flexible bladder 120, the baffle structure may guide the urine through the baffle structure to a bottom region of the flexible bladder 120 (or throughout the flexible bladder 120) to prevent or minimize the splashing noise in the canister. Some of the embodiments of baffle structures described herein may be removable from the flexible bladder 120 and reusable in a different flexible bladder 120. In some embodiments, the baffle structures may be integrally formed (e.g., molded) with the flexible bladder 120. In some embodiments, the flexible bladder 120 with the baffle structure therein may be disposable.

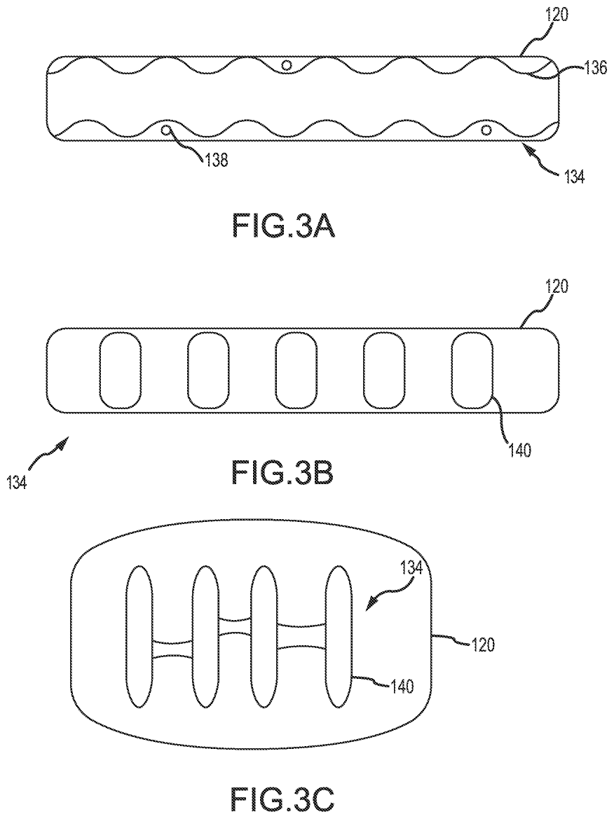

FIG. 3A is a cross-sectional view of an interior of the flexible bladder 120 showing a baffle structure 134 for urine collection system 100, according to an embodiment. The baffle structure 134 may include one or more baffles. For example, the one or more baffles of the baffle structure 134 may include at least one ridge 136 disposed on an interior surface of the bladder 120. The baffle structure 134 may be monolithically formed of the same material as the flexible bladder 120 and/or expandable pouch 121. In some embodiments, the ridge 136 can include a range of lengths extending from the interior surface of the flexible bladder 120 towards the interior. The baffle structure 134 may include an expandable material. In some embodiments, the baffle structure 134 may include a flexible material. The one or more baffles (e.g. ridge 136) can include a textured surface configured to keep the bladder 120 open enough to allow the vacuum 116 to draw the fluid into the bladder 120. In some embodiments, ridge 136 can include a plurality of holes 138 extending there through. The ridge 136 may be sized such that the outer periphery of the ridge 136 interfaces or is adjacent to the interior surface of the opposite surface, whether upper or lower surface, or is spaced from the opposite surface of the flexible bladder 120.

In use, when urine or other fluid enters the interior region of the flexible bladder 120 through the collection connector 124 on the port 122. The urine may expand the pouch 121 until the urine expands the pouch 121 to the maximum allowed without interfering with the operations of the pump 116. The flexible bladder 120 may then be disconnected from the pump 116 and/or the fluid collection device 112 and discarded. The baffles cause to urine to flow in a tortuous path, and thus minimizes sloshing sounds as the urine moves through the pouch 121, and thus reduces or prevents a splashing noise in the flexible bladder 120 as the urine is collected. The baffle structure 134 also may prevent sloshing and splashing of urine held in the flexible bladder 120 as the bladder 120 is moved.

Turning to FIGS. 3B-3C, in some embodiments, the baffle structure 134 may include a foam structure 140 disposed within the bladder. In some embodiments, the foam structure 140 may be removably positionable in the interior region of the flexible bladder 120. In some embodiments, the foam structure 140 can include a non-rigid open-cell foam. In some embodiments, the foam structure 140 can include a non-absorbent cellulose foam. In some embodiments, the foam structure 140 can be compressed prior to wetting with fluid and can expand as the foam structure 140 is wetted. In some embodiments, the foam structure 140 can include a series of discrete segments as shown in FIG. 3B. In other embodiments, the foam structure 140 can fill the interior of the expandable pouch 121 and/or the flexible bladder 120. In some embodiments, the foam structure 140 include a flexible material.

In use, urine entering the flexible bladder 120 may pass through the port 122 and go through a tortuous path through and/or around the baffle structure 134. The baffle structure 134 reduces the distance of urine moving through the flexible bladder 120, and thus reduces or prevents a splashing noise in the bladder 120 as the urine is collected. The expandable pouch 121 also prevents or inhibits sloshing and splashing noises of urine in the flexible bladder 120 as the bladder 120 is moved.

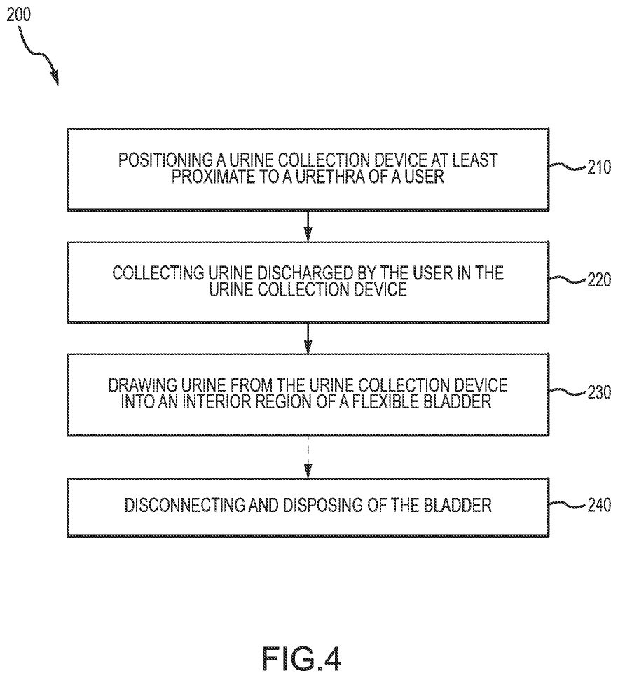

FIG. 4 is a flow diagram of a method 200 for collecting urine, according to an embodiment. The method 200 includes an act 210 of positioning a fluid collection device at least proximate to a urethra of a user, the urine collection device being in fluid communication with the flexible bladder through a conduit. In some embodiments, the method 200 includes an act 220 of collecting urine discharged by the user in the urine collection device. In some embodiments, the method 200 includes an act 230 of drawing urine from the urine collection device through the conduit into an interior region of the bladder, the urine contacting at least a portion of the baffle structure in the interior region of the bladder.

The baffle structure in the method 200 may include any baffle structure described herein. In some embodiments, the one or baffles of the baffle structure of the method 200 may include a foam structure disposed within the bladder. In some embodiments, the one or more baffles of the baffle structure of the method 200 may include one or more ridges disposed on an interior surface of the bladder.

In some embodiments, the method 200 may include an act 240 of disconnecting the conduit from the bladder and disposing of the bladder. In other embodiments, the bladder can be cleaned and reused. In some embodiments, drawing urine from the urine collection device into an interior region of the flexible bladder may utilize a pump connected to the bladder. Prior to disconnecting the conduit from the bladder, the pump of the may be de-energized and/or disconnected.

The acts of the method 200 described above are for illustrative purposes. For example, the acts of the method 200 can be performed in different orders, split into multiple acts, modified, supplemented, or combined. In an embodiment, one or more of the act of the method 200 can be omitted from the method 200. Any of the acts of the method 200 can include using any of the baffle structures disclosed herein.

As used herein, the term “about” or “substantially” refers to an allowable variance of the term modified by “about” or “substantially” by ±10% or ±5%. Further, the terms “less than,” “or less,” “greater than,” “more than,” or “or more” include, as an endpoint, the value that is modified by the terms “less than,” “or less,” “greater than,” “more than,” or “or more.”

While various aspects and embodiments have been disclosed herein, other aspects and embodiments are contemplated. The various aspects and embodiments disclosed herein are for purposes of illustration and are not intended to be limiting.

What is claimed is:

1. A urine collection system, comprising:
a urine collection device configured to be positioned at least proximate to a urethra of a user;
a conduit in fluid communication with the urine collection device;
a flexible bladder having an interior region in fluid communication with the conduit; and
a baffle structure having one or more baffles positioned or positionable in the interior region of the bladder.

2. The urine collection system of claim 1, wherein the bladder includes an expandable pouch.

3. The urine collection system of claim 1, wherein the one or more baffles of the baffle structure includes a ridge disposed on an interior surface of the bladder.

4. The urine collection system of claim 1, wherein the one or more baffles of the baffle structure includes a foam structure disposed within the bladder.

5. The urine collection system of claim 4, wherein the foam structure includes a non-rigid open-cell foam.

6. The urine collection system of claim 4, wherein the foam structure includes a non-absorbent cellulose foam.

7. The urine collection system of claim 1, further comprising a pump in fluid communication with the bladder and configured to pull an at least partial vacuum on the interior region of the canister effective to draw urine from the urine collection device through the conduit into the bladder.

8. The urine collection system of claim 1, further comprising a port secured or securable to the bladder, the port including a collection connector and a vacuum connector.

9. The urine collection system of claim 1, wherein the conduit comprises an interior diameter between about 0.3 inches and about 0.2 inches.

10. A urine collection container, comprising:
a flexible bladder having an interior region;
a baffle structure having one or more baffles disposed within the interior region of the bladder; and
a port secured or securable to the bladder, the port including a collection connector and a vacuum connector.

11. The urine collection container of claim 10, wherein the vacuum connector includes a membrane configured to prevent fluid from traversing the vacuum connector.

12. The urine collection container of claim 10, wherein the port is positioned generally on an upper exterior surface of the bladder.

13. The urine collection container of claim 10, wherein the baffle structure includes ridges extending from an interior surface of the bladder, wherein the ridges and the bladder are monolithically formed from the same material.

14. The urine collection container of claim 10, wherein the bladder and the baffle structure include a flexible material.

15. The urine collection container of claim 10, wherein the bladder includes at least one of a thermoplastic elastomer or silicone rubber.

16. The urine collection container of claim 10, wherein the baffle structure includes a non-rigid open-cell foam.

17. A method of collecting urine, the method comprising:
positioning a urine collection device at least proximate to a urethra of a user, the urine collection device being in fluid communication with a flexible bladder through a conduit;
collecting urine discharged by the user in the urine collection device; and
drawing urine from the urine collection device into an interior region of the flexible bladder, the bladder including a baffle structure having one or more baffles positioned or positionable in the interior region of the bladder, wherein the urine contacts at least a portion of the baffle structure in the interior region of the bladder.

18. The method of claim 17, wherein the one or more baffles of the baffle structure includes a foam structure disposed within the bladder.

19. The method of claim 17, wherein the one or more baffles of the baffle structure includes a ridge disposed on an interior surface of the bladder.

20. The method of claim 17, further comprising disconnecting the conduit from the bladder and disposing of the bladder.
